(19) United States
(12) Patent Application Publication
(10) Pub. No.: US 2021/0283051 A1
(43) Pub. Date: Sep. 16, 2021

(54) VE-PTP INHIBITION IN GLAUCOMA

(71) Applicant: Mannin Research, Inc., Toronto (CA)

(72) Inventor: Susan QUAGGIN, Chicago, IL (US)

(73) Assignee: Mannin Research, Inc., Toronto, ON (CA)

(21) Appl. No.: 17/260,073

(22) PCT Filed: Jul. 12, 2019
(86) PCT No.: PCT/IB2019/055985

§ 371 Date: Jan. 13, 2021

Related U.S. Application Data
(60) Provisional application No. 62/698,017, filed on Jul. 13, 2018.

(51) Int. Cl. A61K 9/00; A61P 27/06; A61K 45/06

(52) U.S. Cl. CPC A61K 9/0048; A61K 45/06; A61P 27/06

(57) ABSTRACT

The disclosure relates to glaucoma, and more particularly to a mouse model of a VE-PTPlacZ mice bred to a Tie2 haploinsufficient mice and the use of VE-PTP inhibition for neuroprotection of glaucoma symptoms of elevated intraocular pressure. There is a method of producing a mouse model through deletion of a single PTPRB allele in a Tek haploinsufficient mouse. Further, the use of VE-PTP inhibition in the limbal vascular plexus provides neuroprotection from glaucoma symptoms of elevated intraocular pressure.

### FIELD OF THE INVENTION

The disclosure relates to glaucoma, and more particularly to a mouse model of a VE-PTPlacZ mice bred to a Tie2 haploinsufficient mice and the use of VE-PTP inhibition for neuroprotection and relief of other glaucoma symptoms resultant from elevated intraocular pressure.

### BACKGROUND OF THE INVENTION

The second leading cause of irreversible blindness worldwide, glaucoma is a devastating disease with no cure. Elevated intraocular pressure (IOP), mainly caused by defects in the aqueous humor outflow pathway, is an important risk factor for disease progression. Reductions in aqueous humor outflow (AHO) lead to altered fluid homeostasis in the anterior chamber, leading to ocular hypertension, retinal ganglion cell death (RGC) and glaucoma.

The majority of AHO is through the conventional route comprised of the trabecular meshwork (TM), and the large, lymphatic-like Schlemm's canal (SC) located in the iridocorneal angle. Aqueous humor from the anterior chamber enters SC through the TM and is drained through a series of collector channels into the episclaral veins. Recent studies have identified the importance of the Angpt-TEK signaling pathway in SC development and maintenance, and loss of function mutations in the Angpt receptor TEK (Tunica interna endothelial cell kinase, also known as Tie2) and its primary ligand ANGPT1 have been identified in patients with primary congenital glaucoma, a severe form of glaucoma characterized by early/childhood onset, buphthalmos and optic neuropathy. Tek knockout mice completely lack SC and exhibit a rapidly progressing glaucoma-like phenotype.

A mouse model is described in U.S. Pat. No. 9,719,135 in which double Angiopoiein 1/Angiopoietin 2 (“Angpt 1/Angpt 2”) knockout mice and Tie 2 knockout mice develop buphthalmos due to elevated intraocular pressure. Both Angpt 1/Angpt 2 double knockout mice and Tie2 knockout mice lack Schlemm's canal. Angiopoietin signaling has a dose-dependent effect on Schlemm's canal formation. Tie2 signaling (activation) has a dose-dependent effect on Schlemm's canal formation. Tie2 activation promotes canalogenesis in the Schlemm's canal, and factors which activate Tie2 include vascular endothelial-phosphotyrosine phosphatase (“VE-PTP”) inhibitors.

Angiopoietin-TEK signaling is essential for development of the lymphatic-like Schlemm's canal, a unique vessel in the ocular anterior chamber. Knockout mice lacking TEK or the Angiopoietin ligands ANGPT1 and ANGPT2 rapidly develop ocular hypertension, buphthalmos and glaucomatous neuropathy.

A similar effect is observed in mice lacking the Angiopoietin ligands ANGPT1 or ANGPT2, confirming that Angiopoietin-TEK signaling is required for SC development.

PCT Patent Application No. PCT/CA2017/000120 entitled “VE-PTP Knockout” filed May 4, 2017 and published as WO2017/190222, incorporated herein by reference in its entirety, described a method of producing a mouse with reduced VE-PTP and Tie2 expression, comprising replacing a single wild type VE-PTP allele with a VE-PTP-null allele and at least one wild-type Tie2-allele with a Tie2-null allele in the mouse's genome. The introduction of a single VE-PTP null allele into a Tie2 heterozygous null mouse genome was shown to decrease phenotypic expression of increased intraocular pressure in the resulting mouse relative to the intraocular pressure of a Tie2 heterozygous null mouse. Also described in WO2017/190222 is a mouse whose genome comprises a VE-PTP null allele, a VE-PTP wild-type allele, two Angiopoietin 1 null alleles, and two Angiopoietin 2 null alleles, wherein the Angiopoietin 1 and/or the Angiopoietin 2 null alleles are conditional null alleles, wherein the conditional null alleles are induced by expressing Cre recombinase, and the mouse has normal intraocular pressure.

Disruption of the angiopoietin-TEK (also known as Tie2) signaling pathway in humans and mice leads to loss of Schlemm's canal, elevated IOP and glaucoma. Partial disruption of the pathway in a TEK heterozygous mouse model reveals a dose-dependent role in SC development, and heterozygous eyes are characterized by a hypoplastic SC with focal narrowing, gaps and convolutions. This misshapen canal is insufficient for normal fluid drainage, and heterozygous mice exhibit elevated IOP (15.39 mmHg) compared to control (12.30 mmHg, p=0.0046).

Indeed, Tek haploinsufficient mice (Tek^{+/-} mice) exhibit defects in SC and TM development with moderate elevation of IOP, indicating a clear dose-dependent effect of TEK signaling in development and function of the aqueous outflow pathway.

Ectopic activation of the TEK receptor can be achieved in vitro and in vivo either by increasing availability of the ANGPT ligands, or by suppression of the phosphatase PTPRB (also known as the Vascular Endothelial Protein Tyrosine Phosphatase, VE-PTP), which strongly dephosphorylates TEK. PTPRB inhibition results in increased TEK phosphorylation at all phosphorylated tyrosine residues and leads to a dramatic increase in downstream signaling.

### SUMMARY OF THE DISCLOSURE

Several of the various features of the disclosure will be described hereinafter. It is to be understood that the invention is not limited in its application to the details set forth in the following embodiments, claims, description and figures. The invention is capable of other embodiments and of being practiced or carried out in numerous other ways. Some of the embodiments are as follows:
1. A method of reducing ocular hypertension in a subject in need thereof, comprising administering to the subject a therapeutically effective amount of a VE-PTP inhibitor.
2. A method of treating glaucoma in a subject in need thereof, comprising administering the subject a compound that causes vasorelaxation of the smooth muscle cells in the limbal vascular plexus, SVP, and/or episclaral veins.
3. The method of embodiment 2, wherein the compound is a VE-PTP inhibitor.
4. A method of treating ocular neuropathy or protecting neuronal cells, including RGCs, from damage due to increased intraocular pressure in a subject in need thereof, comprising administering to the subject a VE-PTP inhibitor.
5. A method of activating Tie2 signaling in the SVP/SCP of a subject in need thereof, the method comprising administering to the patient a VE-PTP inhibitor.
6. A method of activating Tie2 signaling in the limbal vascular plexus of a subject in need thereof, the method comprising administering to the subject a VE-PTP inhibitor.
7. The method according to any one of the above embodiments, wherein the VE-PTP inhibitor is administered to the eye.
8. The method according to any one of the above embodiments, wherein the VE-PTP inhibitor is administered systemically.
9. The method according to any one of the above embodiments, wherein the VE-PTP inhibitor is a small molecule or a biologic.
10. A method of treating glaucoma in a subject in need thereof, comprising administering to the subject a therapeutically effective amount of a VE-PTP inhibitor.
11. A method of reducing ocular hypertension in a subject in need thereof, comprising administering the subject a compound that causes vasorelaxation of the smooth muscle cells in the limbal vascular plexus, SVP, and/or episclaral veins.
12. The method of embodiment 11, wherein the compound is a VE-PTP inhibitor.

### BRIEF DESCRIPTION OF THE FIGURES

The drawings depict only example embodiments of the present disclosure and do not therefore limit its scope. They serve to add specificity and detail.

The patent or application file contains at least one drawing executed in color. Copies of this patent or patent application publication with color drawings will be provided by the Office upon request and payment of the necessary fee.

FIG. 1 shows an exemplary Western Blot analysis, and corresponding graphical representation, of TEK activation in vivo in control mice and VE-PTP/Ptprb heterozygous mice.

FIG. 2 shows a graph of intraocular pressure in control wild-type mice, TEK haploinsufficient, and TEK haploinsufficient/VE-PTP heterozygous mice.

FIG. 3 shows retinal whole mounts stained with anti-BRN3B antibody (which identify RGCs) and corresponding graph comparing retinal ganglion cell loss in TEK haploinsufficient mice and rescue of RGCs in TEK/VE-PTP double heterozygous mice.

FIG. 4 shows a comparison of morphology of Schlemm's canal and corresponding graphical representation of the area and convolutions in wild-type control mice, TEK and VE-PTP haploinsufficient mice, and TEK/VE-PTP double heterozygous mice.

FIG. 5 shows graphs comparing intraocular pressure with the quantity of Schlemm's canal morphological defects and Schlemm's canal area in TEK/VE-PTP double heterozygous mice

FIG. 6 shows immunofluorescent staining of Schlemm's canal endothelium compared to SVP (FSP corresponds to full thickness picture including the SC and the superficial capillary plexus.

### DETAILED DESCRIPTION

In an embodiment of the present disclosure there is a mouse model of a VE-PTPlacZ mice bred to a Tie2 haploinsufficient mice.

In an embodiment of the present disclosure there is a method of producing a mouse model through deletion of a single PTPRB allele in a Tek haploinsufficient mouse.

In an embodiment of the present disclosure, the use of VE-PTP inhibition in the limbal vascular plexus/superficial capillary plexus provides neuroprotection from (glaucoma symptoms of) elevated intraocular pressure.

In an embodiment of the present disclosure there is mouse whose genome comprises a deletion of a single PTPRB allele in a Tek haploinsufficient mouse.

In an embodiment of the present disclosure there is method of treating glaucoma through VE-PTP inhibition in the limbal vascular plexus for neuroprotection from (glaucoma symptoms of) elevated intraocular pressure.

In an embodiment of the present disclosure there is a Tek haploinsufficient mouse model in which deleting a single PTPRB allele is sufficient to increase TEK activation, reduce intraocular pressure (IOP) and suppress pressure-related loss of retinal ganglion cells (RGC). The result is RGC protection with no change in the Schlemm's Canal (SC) canal diameter.

In an embodiment of the present disclosure there is a mouse model of VE-PTPlacZ mice bred to Tie2 haploinsufficient mice for measuring neuroprotection of glaucoma symptoms of elevated intraocular pressure.

In a further embodiment of the present disclosure, the use of VE-PTP/PTPRB inhibition provides an IOP-lowering treatment strategy for patients with glaucoma.

In a further embodiment of the present disclosure, the use of VE-PTP/PTPRB inhibition provides increased aqueous humour outflow downstream of the Schlemm's canal.

In a further embodiment of the present disclosure, vasorelaxation of smooth muscles around the superficial capillary plexus in the eye provides neuroprotection and spares loss of retinal ganglion cells caused by high IOP.

In a further embodiment of the present disclosure, pharmacological TEK activation can be used for treatment of high intraocular pressure and glaucoma.

In an embodiment of this disclosure, there is a method of protecting against RGC loss in glaucoma and for IOP lowering in Tie2/TEK haploinsufficient mice with addition of haploinsufficiency of VE-PTP.

Aqueous humor outflow is limited by the flow capacity and resistance of the SC inner wall and reductions in SC area are assumed to have a direct effect on outflow capacity. However, flow resistance of the SC inner wall is not the only factor affecting aqueous humor outflow, and as described by the Goldmann equation (equation 1) both outflow and IOP are directly related to the episclaral venous pressure (EVP). The data presented herein suggest that ptprb inhibition increases outflow downstream of Schlemm's canal allowing larger vessels and more outflow. This, is turn, is neuroprotective, protecting RGCs.

IOP=F/C+EVP Equation 1:

Without wanting to be bound by proposed mechanisms, the reduced IOP obtained by rescue of TEK haploinsuficient mice with VE-PTP deletion is likely due to vasodilation of draining vessels, including the superficial vascular plexus, since before the deletion it expresses high levels of PTPRB. By deletion of a single VE-PTP allele, (systemic) TEK activation was increased and there was rescue of the ocular hypertension and RGC loss observed in Tek heterozygous null mice.

The mechanism of ocular protection through VEPTP inhibition appears to be through the effect of VE-PTP inhibition on the superficial venous plexus, which normally expresses VE-PTP.

The results suggest that the Schlemm's canal itself does not express the Beta-gal VE-PTP reporter, suggesting that VE-PTP is not in SC itself, or, if it is, it is present at lower levels than the surrounding blood vasculature. VE-PTP does not appear to be expressed during development in the early capillary plexus that gives rise to the Schlemm's canal but it appears that the progenitors express VE-PTP.

IOP lowering and neuroprotection can be achieved through VE-PTP inhibition, likely through enhanced outflow by direct effect of VE-PTP inhibition on the draining vessels (not necessarily in the Schlemm's itself).

Using a VE-PTP-LacZ reporter mouse strain, it was determined that there is VE-PTP expression in the limbal vascular plexus which drains aqueous humor from SC, and which sprouts during development to form the canal. However, VE-PTP expression was not identified in the mature SC endothelium itself. Double TEK;VE-PTP heterozygous mice were generated and, unlike TEK heterozygous littermates, TEK;VE-PTP double-heterozygous mice had normal IOP and did not develop the ocular disease phenotype of TEK deficient mice. It was determined that that TEK activation through inhibition of this negative regulator (VE-PTP) provides a treatment strategy for patients with congenital glaucoma.

The following are additional embodiments of the invention:

1. A method of reducing ocular hypertension in a subject in need thereof, comprising administering to the subject a therapeutically effective amount of a VE-PTP inhibitor.

2. A method of treating glaucoma in a subject in need thereof, comprising administering the subject a compound that causes vasorelaxation of the smooth muscle cells in the limbal vascular plexus, SVP, and/or episclaral veins.

3. The method of embodiment 2, wherein the compound is a VE-PTP inhibitor.

4. A method of treating ocular neuropathy or protecting neuronal cells, including RGCs, from damage due to increased intraocular pressure in a subject in need thereof, comprising administering to the subject a VE-PTP inhibitor.

5. A method of activating Tie2 signaling in the SVP/SCP of a subject in need thereof, the method comprising administering to the patient a VE-PTP inhibitor.

6. A method of activating Tie2 signaling in the limbal vascular plexus of a subject in need thereof, the method comprising administering to the subject a VE-PTP inhibitor.

7. The method according to any one of the above embodiments, wherein the VE-PTP inhibitor is administered to the eye.

8. The method according to any one of the above embodiments, wherein the VE-PTP inhibitor is administered systemically.

9. The method according to any one of the above embodiments, wherein the VE-PTP inhibitor is a small molecule or a biologic.

10. A method of treating glaucoma in a subject in need thereof, comprising administering to the subject a therapeutically effective amount of a VE-PTP inhibitor.

11. A method of reducing ocular hypertension in a subject in need thereof, comprising administering the subject a compound that causes vasorelaxation of the smooth muscle cells in the limbal vascular plexus, SVP, and/or episclaral veins.

12. The method of embodiment 11, wherein the compound is a VE-PTP inhibitor.

In an embodiment, the VE-PTP inhibitor is a small molecule. In another embodiment, the VE-PTP inhibitor is a biologic. Examples of VE-PTP inhibitors are well know in the art, including those described in the following articles:
Antioxid Redox Signal. 2014 May 10; 20(14):2130-40. doi: 10.1089/ars.2013.5463. Epub 2014 Feb. 4. Hydroxyindole carboxylic acid-based inhibitors for receptor-type protein tyrosine protein phosphatase beta. Zeng L F, Zhang R Y, Bai Y, Wu L, Gunawan A M, Zhang Z Y. PMID: 24180557 PMCID: PMC3995206 DOI: 10.1089/ars.2013.5463
J Biol Chem. 2004 Jun. 4; 279(23):24226-35. Epub 2004 Mar. 15. Structure-based design of selective and potent inhibitors of protein-tyrosine phosphatase beta. Lund I K, Andersen H S, Iversen L F, Olsen O H, Møller K B, Pedersen A K, Ge Y, Holsworth D D, Newman M J, Axe F U, Møller N P. PMID: 15024017 DOI: 10.1074/jbc.M313027200
Proc Natl Acad Sci USA. 2006 Jul. 11; 103(28):10606-11. Epub 2006 Jun. 29. Discovery of protein phosphatase inhibitor classes by biology-oriented synthesis. Nören-Müller A, Reis-Corréa I Jr, Prinz H, Rosenbaum C, Saxena K, Schwalbe H J, Vestweber D, Cagna G, Schunk S, Schwarz O, Schiewe H, Waldmann H. PMID: 16809424 PMCID: PMC1502279 DOI: 10.1073/pnas.0601490103
J Inorg Biochem. 2003 Aug. 1; 96(2-3):321-30. Mechanism of insulin sensitization by BMOV (bis maltolato oxo vanadium); unliganded vanadium (VO4) as the active component. Peters K G, Davis M G, Howard B W, Pokross M, Rastogi V, Diven C, Greis K D, Eby-Wilkens E, Maier M, Evdokimov A, Soper S, Genbauffe F. PMID: 12888267
Am J Physiol Heart Circ Physiol. 2004 July; 287(1):H268-76. Epub 2004 Feb. 26. Tyrosine phosphatase inhibition augments collateral blood flow in a rat model of peripheral vascular disease. Carr A N, Davis M G, Eby-Wilkens E, Howard B W, Towne B A, Dufresne T E, Peters K G. PMID: 14988069 DOI: 10.1152/ajpheart.00007.2004
Angiogenesis. 2009; 12(1):25-33. doi: 10.1007/s10456-008-9126-0. Epub 2009 Jan. 1. Tyrosine phosphatase beta regulates angiopoietin-Tie2 signaling in human endothelial cells. Yacyshyn O K, Lai P F, Forse K, Teichert-Kuliszewska K, Jurasz P, Stewart D J. PMID: 19116766 DOI: 10.1007/s10456-008-9126-0
J Biol Chem. 2012 Mar. 16; 287(12):9322-6. doi: 10.1074/jbc.C111.320796. Epub 2012 Jan. 24. Picomolar concentrations of free zinc(II) ions regulate receptor protein-tyrosine phosphatase R activity. Wilson M, Hogstrand C, Maret W. PMID: 22275360 PMCID: PMC3308757 DOI: 10.1074/jbc.C111.320796
J Med Chem. 2009 Nov. 12; 52(21):6649-59. doi: 10.1021/jm9008899. Knowledge-based characterization of similarity relationships in the human protein-tyrosine phosphatase family for rational inhibitor design. Vidovid D, Schurer S C. PMID: 19810703 PMCID: PMC2786264 DOI: 10.1021/jm9008899
FEBS Lett. 2004 May 21; 566(1-3):35-8. Bisperoxovanadium compounds are potent PTEN inhibitors. Schmid A C, Byrne R D, Vilar R, Woscholski R. PMID: 15147864 DOI: 10.1016/j.febslet.2004.03.102
Bioorg Med Chem. 2012 Jul. 15; 20(14):4371-6. doi: 10.1016/j.bmc.2012.05.040. Epub 2012 May 24. Arylstibonic acids are potent and isoform-selective inhibitors of Cdc25a and Cdc25b phosphatases. Mak L H, Knott J, Scott K A, Scott C, Whyte G F, Ye Y, Mann D J, Ces O, Stivers J, Woscholski R. PMID: 22705189 PMCID: PMC3389297 DOI: 10.1016/j.bmc.2012.05.040
ACS Chem Biol. 2006 Dec. 15; 1(12):780-90. A small molecule inhibitor for phosphatase and tensin homologue deleted on chromosome 10 (PTEN). Rosivatz E, Matthews J G, McDonald N Q, Mulet X, Ho K K, Lossi N, Schmid A C, Mirabelli M, Pomeranz K M, Erneux C, Lam E W, Vilar R, Woscholski R. PMID: 17240976 DOI: 10.1021/cb600352f
J Med Chem. 2013 Jun. 27; 56(12):4990-5008. doi: 10.1021/jm400248c. Epub 2013 Jun. 6. A potent and selective small-molecule inhibitor for the lymphoid-specific tyrosine phosphatase (LYP), a target associated with autoimmune diseases. He Y, Liu S, Menon A, Stanford S, Oppong E, Gunawan A M, Wu L, Wu D J, Barrios A M, Bottini N, Cato A C, Zhang Z Y. PMID: 23713581 PMCID: PMC3711248 DOI: 10.1021/jm400248c
J Biol Chem. 2009 Apr. 24; 284(17):11385-95. doi: 10.1074/jbc.M807241200. Epub 2009 Feb. 20. GLEPP1/protein-tyrosine phosphatase phi inhibitors block chemotaxis in vitro and in vivo and improve murine ulcerative colitis.
Gobert R P, van den Eijnden M, Szyndralewiez C, Jorand-Lebrun C, Swinnen D, Chen L, Gillieron C, Pixley F, Juillard P, Gerber P, Johnson-Leger C, Halazy S, Camps M, Bombrun A, Shipp M, Vitte P A, Ardissone V, Ferrandi C, Perrin D, Rommel C, Hooft van Huijsduijnen R. PMID: 19233845 PMCID: PMC2670144 DOI: 10.1074/jbc.M807241200
Bioorg Med Chem. 2007 Aug. 1; 15(15):5137-49. Epub 2007 May 17. 5-Arylidene-2,4-thiazolidinediones as inhibitors of protein tyrosine phosphatases. Maccari R, Paoli P, Ottani R, Jacomelli M, Ciurleo R, Manao G, Steindl T, Langer T, Vigorita M G, Camici G. PMID: 17543532 DOI: 10.1016/j.bmc.2007.05.027
Molecules. 2018 Mar. 2; 23(3). pii: E569. doi: 10.3390/molecules23030569. Targeting Receptor-Type Protein Tyrosine Phosphatases with Biotherapeutics: Is Outside-in Better than Inside-Out?Senis Y A, Barr A J. PMID: 29498714 DOI: 10.3390/molecules23030569

This is a comprehensive review of inhibitors targeting tyrosine phosphatases:
Trends Pharmacol Sci. 2017 June; 38(6):524-540. doi: 10.1016/j.tips.2017.03.004. Epub 2017 Apr. 12. Targeting Tyrosine Phosphatases: Time to End the Stigma. Stanford S M, Bottini N. PMID: 28412041 PMCID: PMC5494996 DOI: 10.1016/j.tips.2017.03.004
Curr Diab Rep. 2016 December; 16(12):126. Targeting Tie2 for Treatment of Diabetic Retinopathy and Diabetic Macular Edema. Campochiaro P A, Peters K G. PMID: 27778249 DOI: 10.1007/s11892-016-0816-5
Prog Retin Eye Res. 2015 November; 49:67-81. doi: 10.1016/j.preteyeres.2015.06.002. Epub 2015 Jun. 23. Molecular pathogenesis of retinal and choroidal vascular diseases. Campochiaro P A. PMID: 26113211 PMCID: PMC4651818 DOI: 10.1016/j.preteyeres.2015.06.002

Examples of VE-PTP inhibitors that are biologics include antibodies and the following drugs:
J Exp Med. 2015 Dec. 14; 212(13):2267-87. doi: 10.1084/jem.20150718. Epub 2015 Dec. 7. Interfering with VE-PTP stabilizes endothelial junctions in vivo via Tie-2 in the absence of VE-cadherin. Frye M, Dierkes M, Kuppers V, Vockel M, Tomm J, Zeuschner D, Rossaint J, Zarbock A, Koh G Y, Peters K, Nottebaum A F, Vestweber D. PMID: 26642851 PMCID: PMC4689167 DOI: 10.1084/jem.20150718
J Clin Invest. 2014 October; 124(10):4564-76. doi: 10.1172/JCI74527. Epub 2014 Sep. 2. Targeting VE-PTP activates TIE2 and stabilizes the ocular vasculature. Shen J, Frye M, Lee B L, Reinardy J L, McClung J M, Ding K, Kojima M, Xia H, Seidel C, Lima e Silva R, Dong A, Hackett S F, Wang J, Howard B W, Vestweber D, Kontos C D, Peters K G, Campochiaro P A. PMID: 25180601 PMCID: PMC4191011 DOI: 10.1172/JC174527
J Cell Biol. 2009 May 18; 185(4):657-71. doi: 10.1083/jcb.200811159. VE-PTP controls blood vessel development by balancing Tie-2 activity. Winderlich M, Keller L, Cagna G, Broermann A, Kamenyeva O, Kiefer F, Deutsch U, Nottebaum A F, Vestweber D. PMID: 19451274 PMCID: PMC2711575 DOI: 10.1083/jcb.200811159

Examples of drugs that promote relaxation of smooth muscle cells include vasorelaxants/vasodilators such as:
Alpha-adrenoceptor antagonists (alpha-blockers)
Angiotensin converting enzyme (ACE) inhibitors
Angiotensin receptor blockers (ARBs)
Beta2-adrenoceptor agonists (β2-agonists)
Calcium-channel blockers (CCBs)
Centrally acting sympatholytics
Direct acting vasodilators
Endothelin receptor antagonists
Ganglionic blockers
Nitrodilators
Phosphodiesterase inhibitors
Potassium-channel openers
Renin inhibitors

### EXAMPLES

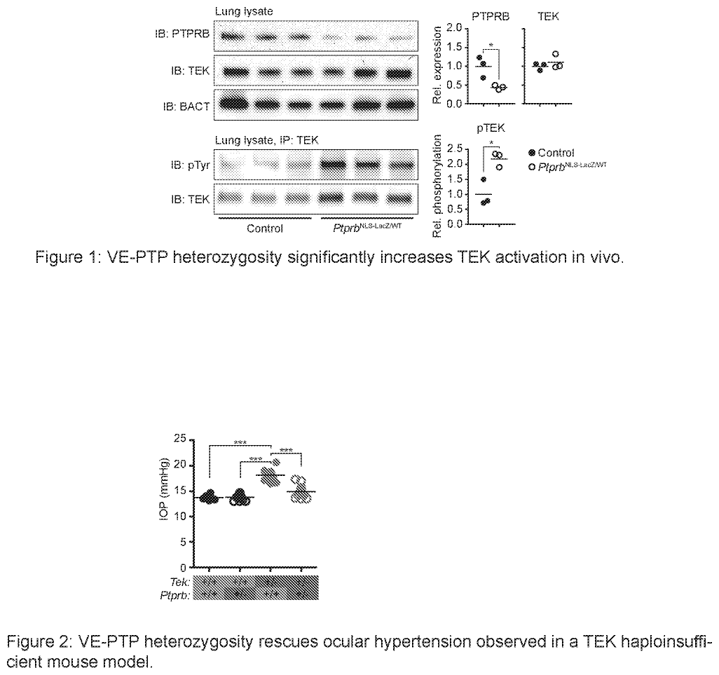

Ptprb haploinsufficient mice have elevated Tek phosphorylation. The ptprb haploinsufficient mouse strain described herein is a VE-PTP-LacZ reporter mouse strain. To elevate the level of TEK phosphorylation in vivo, a previously described Ptprbs-LacZ reporter allele was used to delete a single allele of the Ptprb gene. This construct incorporates a LacZ cDNA tagged with a nuclear localization signal in place of the first exon of Ptprb, preventing production of PTPRB protein. It's known that Ptprb^{NLS-LacZ/WT} mice are born normally, although expression of PTPRB is reduced by approximately 50% (FIG. 1). Total TEK expression was unaffected. As expected, reductions in phosphatase abundance had a direct effect on TEK activation, and Ptprb^{NLS-LacZ/WT} mice showed a significant increase in phosphorylated TEK when measured using an immunoprecipitation assay (FIG. 1).

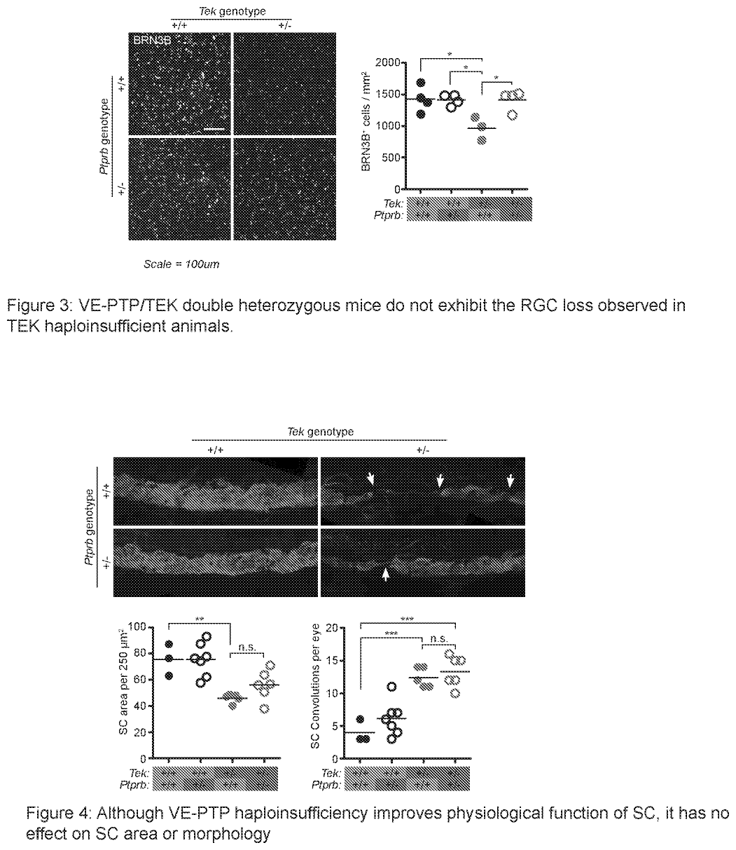

In the present disclosure, deletion of a single Ptprb allele in a Tek haploinsufficient model of ocular hypertension lowers IOP and prevents associated RGC loss. Tek heterozygous mice measured at 30 weeks of age were found to have elevated IOP (FIG. 2, Control: 13.7±0.23, Tek^{+/-}18.15±0.33 mmHg). While Ptprb^{NLS-LacZ/WT} mice had normal IOP (13.81±0.82 mmHg), incorporation of this allele into the Tek^{+/-} model was beneficial and partially rescued the ocular hypertension associated with Tek haploinsufficiency (Tek^{+/-};Ptprb^{NLS-LacZ/WT} IOP: 14.92±0.31 mmHg). To confirm the impact of the Ptprb^{NLS-LacZ}-induced IOP reduction on the retina, BRN3B positive ganglion cells at 19 weeks of age were counted (FIG. 3). While Tek heterozygous mice showed a marked reduction in RGCs, this loss was blunted in Tek^{+/};Ptprb^{NLS-LacZ/WT} mice.

Ptprb heterozygosity does not alter SC morphology. Finding that Ptprb heterozygosity was protective in the Tek^{+/-} murine glaucoma model, Schlemm's canal morphology was examined to determine the mechanism for this protection. CD31 staining followed by confocal microscopy exposes a hypomorphic SC characterized by focal narrowing and convolutions in Tek^{+/-} mice (FIG. 4).

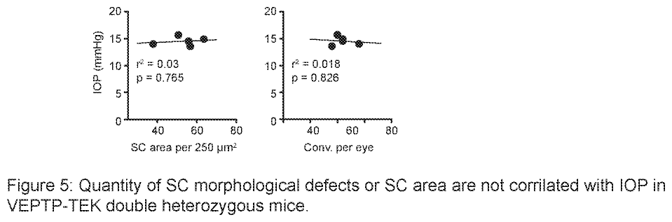

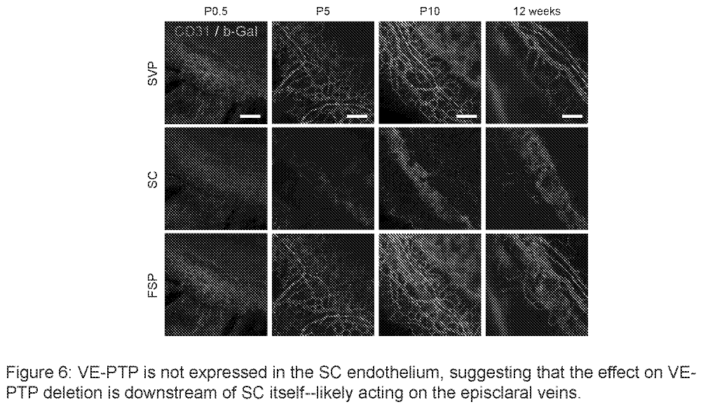

It was determined that although Tek^{+/-};Ptprb^{NLS-LacZ/WT} mice had a lower IOP then Tek^{+/-} littermates, there was no significant increase in SC area or reduction in focal convolutions. While the overall change in SC area was not significant due to inter-animal variability, SC area or number of SC convolutions per eye might be correlated with IOP within each experimental group. However, regression analysis (FIG. 5) revealed no correlation between IOP and either SC area or convolutions per eye in Tek^{+/-};Ptprb^{NLS-LacZ/WT} mice-suggesting that the mechanism lay elsewhere.

Finding no rescue of SC area or morphology in Tek^{+/-};Ptprb^{NLS-LacZ/WT} mice, Ptprb expression in the anterior chamber was examined to seek potential mechanisms for the observed effect on IOP and RGC survival. It was confirmed through whole mount staining of Ptprb^{NLS-LacZ/WT} eyes with anti-CD31 and anti-βgal antibodies (FIG. 6) that in the limbal region, Ptprb expression was activated in the superficial capillary plexus (SVP) between P0.5 and P5, and SVP expression remained high throughout life. However, consistent with the lack of apparent effect on SC morphology or area, Ptprb was not expressed by the SC endothelial cells themselves at any point during development or in adulthood.

It's known that aqueous humor outflow is limited by the flow capacity and resistance of the SC inner wall, and reductions in SC area are assumed to have a direct effect on outflow capacity. However, it was determined that flow resistance of the SC inner wall is not the only factor affecting aqueous humor outflow, and as described by the Goldmann equation (equation 1) both outflow and IOP are directly related to the episclaral venous pressure (EVP).

IOP=F/C+EVP Equation 1:

It was determined that ptprb inhibition (genetic deletion) increases outflow downstream of Schlemm's canal. It is possible that vasorelaxation of smooth muscles around the SVP allows for larger vessels and more outflow, which in turn is neuroprotective resulting in sparing of RGCs.

Angpt-TEK signaling is essential for development and maintenance of Schlemm's canal and dysregulation of this signaling pathway results in glaucoma in mice and humans. In addition to the angiopoietin ligands, activation of the TEK receptor is regulated by the endothelial-specific receptor type phosphatase PTPRB (VE-PTP). It was determined that Ptprb heterozygosity results in (systemic) TEK over activation and provides partial compensation for the developmental phenotypes arising from insufficient TEK signaling in the iridocorneal angle.

FIG. 1 is a gel and corresponding graph of the TEK activation in vivo in control mice and VE-PTP heterozygous mice.

It was determined that Ptprb heterozygosity results in systemic TEK over activation and provides partial compensation for the developmental phenotypes arising from insufficient TEK signaling in the iridocorneal angle. FIG. 2 shows that VE-PTP heterozygosity rescues ocular hypertension observed in a TEK haploinsufficient mouse model. Deletion of a single Ve-ptp allele in mice prevents the ocular hypertension observed in a Tek haploinsufficient model of glaucoma.

FIG. 3 shows that VE-PTP/TEK double heterozygous mice do not exhibit the RGC loss observed in TEK haploinsufficient animals. Compared to Tie2^{+/-} controls, VE-PTP;Tie2 double heterozygous mice were protected from ocular hypertension and showed reduced BRN3B-positive ganglion cell loss at 12 weeks. Representative images from retina whole mounts stained with anti-BRN3B antibody are shown in FIG. 3.

FIG. 4 shows that although VE-PTP haploinsufficiency improves physiological function of SC, it has no effect on SC area or morphology. Although VE-PTP haploinsufficiency increases TEK phosphorylation, lowers intraocular pressure and prevents retinal ganglion cell loss, no effect on Schlemm's canal area or morphology was observed by confocal microscopy

FIG. 5 shows that the quantity of SC morphological defects or SC area do not appear to be correlated with IOP in VEPTP-TEK double heterozygous mice.

FIG. 6 shows that VE-PTP is not expressed in the SC endothelium, and it was determined that the effect on VEPTP deletion on improving drainage through the Schlemm's canal is downstream of SC itself, acting on the superficial capillary plexus and/or episclaral veins. FSP corresponds to full thickness picture including the SC and the superficial capillary plexus.

While embodiments of the disclosure have been described in the detailed description, the scope of the claims should not be limited by the preferred embodiments set forth in the examples, but should be given the broadest interpretation consistent with the description as a whole.

All publications, patents, patent applications and other documents cited in this application are hereby incorporated by reference in their entireties for all purposes to the same extent as if each individual publication, patent, patent application or other document were individually indicated to be incorporated by reference for all purposes.

What is claimed is:

1. A method of reducing ocular hypertension in a subject in need thereof, comprising administering to the subject a therapeutically effective amount of a VE-PTP inhibitor.

2. A method of treating glaucoma in a subject in need thereof, comprising administering the subject a compound that causes vasorelaxation of the smooth muscle cells in the limbal vascular plexus, SVP, and/or episclaral veins.

3. The method of claim 2, wherein the compound is a VE-PTP inhibitor.

4. A method of treating ocular neuropathy or protecting neuronal cells, including RGCs, from damage due to increased intraocular pressure in a subject in need thereof, comprising administering to the subject a VE-PTP inhibitor.

5. A method of activating Tie2 signaling in the SVP/SCP of a subject in need thereof, the method comprising administering to the patient a VE-PTP inhibitor.

6. A method of activating Tie2 signaling in the limbal vascular plexus of a subject in need thereof, the method comprising administering to the subject a VE-PTP inhibitor.

7. The method according to any one of the above claims, wherein the VE-PTP inhibitor is administered to the eye.

8. The method according to any one of the above claims, wherein the VE-PTP inhibitor is administered systemically.

9. The method according to any one of the above claims, wherein the VE-PTP inhibitor is a small molecule or a biologic.

10. A method of treating glaucoma in a subject in need thereof, comprising administering to the subject a therapeutically effective amount of a VE-PTP inhibitor.

11. A method of reducing ocular hypertension in a subject in need thereof, comprising administering the subject a compound that causes vasorelaxation of the smooth muscle cells in the limbal vascular plexus, SVP, and/or episclaral veins.

12. The method of claim 11, wherein the compound is a VE-PTP inhibitor.
